(19) United States
(12) Patent Application Publication
(10) Pub. No.: US 2026/0354792 A1
(43) Pub. Date: Nov. 12, 2026

(54) DIAGNOSTIC IMAGING CATHETER, METHOD, AND DIAGNOSTIC IMAGING APPARATUS

(71) Applicant: TERUMO KABUSHIKI KAISHA, Tokyo (JP)

(72) Inventors: Keishi HASHIMOTO, Fujinomiya Shizuoka (JP); Yasunori YAMASHITA, Fujinomiya Shizuoka (JP); Kenta SUZUKI, Fujinomiya Shizuoka (JP)

(21) Appl. No.: 19/713,570

(22) Filed: Jun. 18, 2026

Related U.S. Application Data
(63) Continuation of application No. PCT/JP2024/041542, filed on Nov. 22, 2024.

(30) Foreign Application Priority Data
Dec. 25, 2023 (JP) 2023-218584

(51) Int. Cl. A61B 8/12; A61B 8/00; A61B 90/00

(52) U.S. Cl. CPC A61B 8/12; A61B 8/4416; A61B 8/445; A61B 90/03; A61B 90/39; A61B 2090/035; A61B 2090/3966

(57) ABSTRACT

A diagnostic imaging catheter includes a drive shaft and an imaging core connected to the drive shaft and including: an ultrasound transmitter and receiver, an optical transmitter and receiver, an optical transmission line connected to the optical transmitter and receiver, a holding member that holds the line, and a housing that supports the ultrasound transmitter and receiver, the optical transmitter and receiver, and the holding member. The housing has an opening portion through which an ultrasound wave transmitted and received by the ultrasound transmitter and receiver and light transmitted and received by the optical transmitter and receiver can pass. Each of the ultrasound transmitter and receiver and the holding member is positioned in a circumferential direction by edge portions on both sides of the opening portion such that the ultrasound transmitter and receiver and the holding member are restrained from rotating relative to the housing in the circumferential direction.

### CROSS-REFERENCE TO RELATED APPLICATION

This application is a continuation of International Patent Application No. PCT/JP2024/041542 filed Nov. 22, 2024, which is based upon and claims the benefit of priority from Japanese Patent Application No. 2023-218584, filed Dec. 25, 2023, the entire contents of which are incorporated herein by reference.

### TECHNICAL FIELD

The present disclosure relates to a diagnostic imaging catheter, a method of manufacturing the diagnostic imaging catheter, and a diagnostic imaging apparatus.

### BACKGROUND ART

Conventionally, a diagnostic imaging catheter used for acquiring an image for diagnosing a diseased site or the like in a living body is known. As this type of diagnostic imaging catheter, dual-type diagnostic imaging catheters using both intravascular ultrasound (IVUS) and optical coherence tomography (OCT: Optical Coherence Tomography, or OFDI: Optical Frequency Domain Imaging) have been developed.

A conventional dual-type diagnostic imaging catheter includes a sheath, a drive shaft, a housing provided at a distal end of the drive shaft, an ultrasound transmitter and receiver and an optical transmitter and receiver supported by the housing, and a positioning member that fixes a relative position of the optical transmitter and receiver with respect to the ultrasound transmitter and receiver. Furthermore, an opening portion through which ultrasound waves and light can pass is formed in the housing.

### SUMMARY

In the conventional dual-type diagnostic imaging catheter, the ultrasound transmitter and receiver and the optical transmitter and receiver are positioned relative to the housing so as to maintain a transmission direction of light from the optical transmitter and receiver in a fixed direction relative to a transmission direction of an ultrasound wave from the ultrasound transmitter and receiver. However, in the conventional dual-type diagnostic imaging catheter, when the ultrasound transmitter and receiver and the optical transmitter and receiver are positioned relative to the housing, the positioning member is easily rotatable in the catheter circumferential direction relative to the housing. Therefore, in the conventional dual-type diagnostic imaging catheter, there is still room for improvement from the viewpoint of ease of operation of positioning the ultrasound transmitter and receiver and the optical transmitter and receiver relative to the housing while maintaining the positional relationship between the transmission direction of the ultrasound wave from the ultrasound transmitter and receiver and the transmission direction of the light from the optical transmitter and receiver in a desired state in the catheter circumferential direction.

Embodiments of the present disclosure provide a diagnostic imaging catheter capable of easily positioning an ultrasound transmitter and receiver and an optical transmitter and receiver relative to a housing while maintaining a positional relationship between a transmission direction of an ultrasound wave from the ultrasound transmitter and receiver and a transmission direction of light from the optical transmitter and receiver in a desired state in a catheter circumferential direction.

In one embodiment, a diagnostic imaging catheter comprises: a drive shaft; and an imaging core connected to the drive shaft and including: an ultrasound transmitter and receiver, an optical transmitter and receiver, an optical transmission line connected to the optical transmitter and receiver, a holding member that holds the optical transmission line, and a housing that supports the ultrasound transmitter and receiver, the optical transmitter and receiver, and the holding member. The housing has an opening portion through which an ultrasound wave to be transmitted and received by the ultrasound transmitter and receiver and light to be transmitted and received by the optical transmitter and receiver can pass. Each of the ultrasound transmitter and receiver and the holding member is positioned in a catheter circumferential direction by edge portions on both sides of the opening portion such that the ultrasound transmitter and receiver and the holding member are restrained from rotating relative to the housing in the catheter circumferential direction.

According to the present disclosure, it is possible to provide a diagnostic imaging catheter capable of easily positioning an ultrasound transmitter and receiver and an optical transmitter and receiver relative to a housing while maintaining a positional relationship between a transmission direction of an ultrasound wave from the ultrasound transmitter and receiver and a transmission direction of light from the optical transmitter and receiver in a desired state in a catheter circumferential direction.

### BRIEF DESCRIPTION OF DRAWINGS

FIG. 1 is a view illustrating a diagnostic imaging apparatus including a diagnostic imaging catheter according to an embodiment.

FIG. 2A is a view illustrating the diagnostic imaging catheter illustrated in FIG. 1 as a single unit, and is a view illustrating a pushed-in state of a probe and an inner tube thereof.

FIG. 2B is a view illustrating the diagnostic imaging catheter illustrated in FIG. 1 as a single unit, and is a view illustrating a retracted state of the probe and the inner tube.

FIG. 3 is a view illustrating a distal end portion of the diagnostic imaging catheter illustrated in FIG. 1.

FIG. 4 is a perspective view of a vicinity of the imaging core illustrated in FIG. 3.

FIG. 5 is a top view of a vicinity of the imaging core illustrated in FIG. 3.

FIG. 6 is a view illustrating a positional relationship among the imaging core, a drive shaft, an electrical signal line, and an optical transmission line in a case where the vicinity of the imaging core illustrated in FIG. 3 is viewed from a side surface side thereof.

FIG. 7 is a cross-sectional view of the probe in a plane orthogonal to a longitudinal direction of the catheter at a holding member thereof.

FIG. 8 is a perspective view of the housing illustrated in FIG. 4.

FIG. 9 is a flowchart illustrating an example of a method for manufacturing the diagnostic imaging catheter illustrated in FIG. 1.

FIG. 10 is a view illustrating an outline of a retraction step illustrated in FIG. 9.

### DETAILED DESCRIPTION

Hereinafter, embodiments of the present disclosure will be described with reference to the drawings. In the drawings, the same components are denoted by the same reference signs.

Hereinafter, in the present disclosure, the longitudinal direction of the diagnostic imaging catheter is referred to as “catheter longitudinal direction A”. Hereinafter, in the present disclosure, in the diagnostic imaging catheter, a tip side to be inserted into a living body in the catheter longitudinal direction A is referred to as a “distal side”, and a hand side that is opposite to the distal side and operated outside the living body in the catheter longitudinal direction A is referred to as a “proximal side”. Hereinafter, in the present disclosure, in the catheter longitudinal direction A, a direction from the proximal side to the distal side of the diagnostic imaging catheter may be simply referred to as an “insertion direction A1”. Hereinafter, in the present disclosure, in the catheter longitudinal direction A, a direction from the distal side toward the proximal side of the diagnostic imaging catheter may be simply referred to as a “withdrawal direction A2”.

Furthermore, hereinafter, in the present disclosure, the circumferential direction around a central axis O of the drive shaft of the diagnostic imaging catheter is referred to as a “catheter circumferential direction B”. Moreover, hereinafter, in the present disclosure, in an arbitrary cross-sectional view of the diagnostic imaging catheter orthogonal to the central axis O of the drive shaft, a radial direction of an imaginary circle centered on the central axis O is referred to as a “catheter radial direction C”.

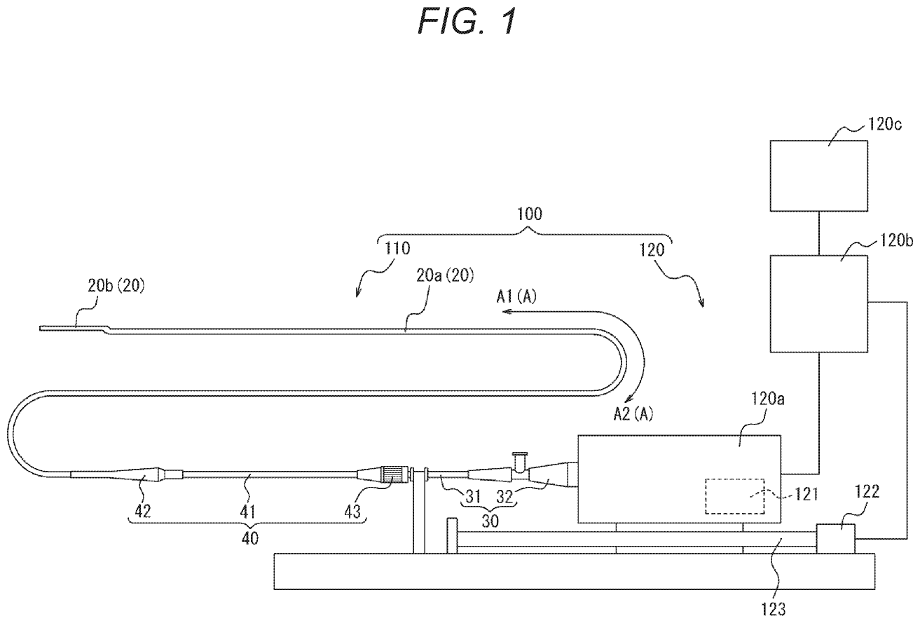

FIG. 1 is a view illustrating a diagnostic imaging apparatus 100 including a diagnostic imaging catheter 110 according to an embodiment. As illustrated in FIG. 1, the diagnostic imaging apparatus 100 includes the diagnostic imaging catheter 110 and an external device 120. FIG. 1 illustrates a state where the diagnostic imaging catheter 110 is connected to the external device 120.

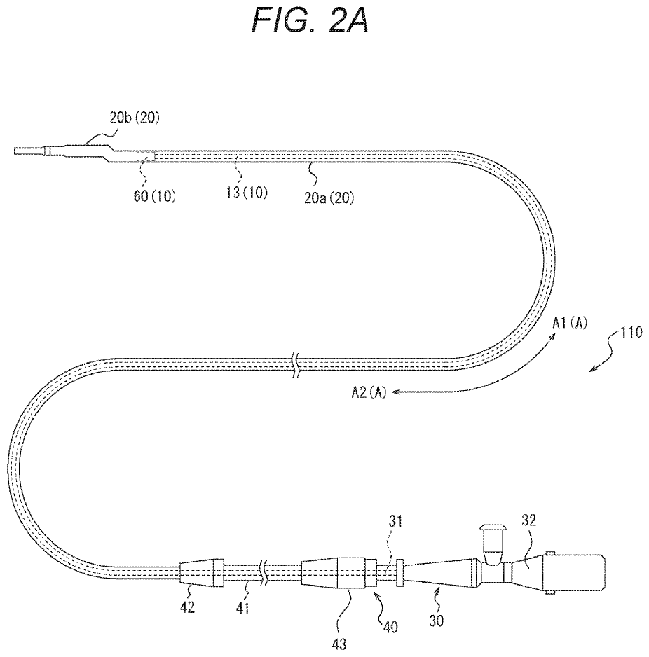

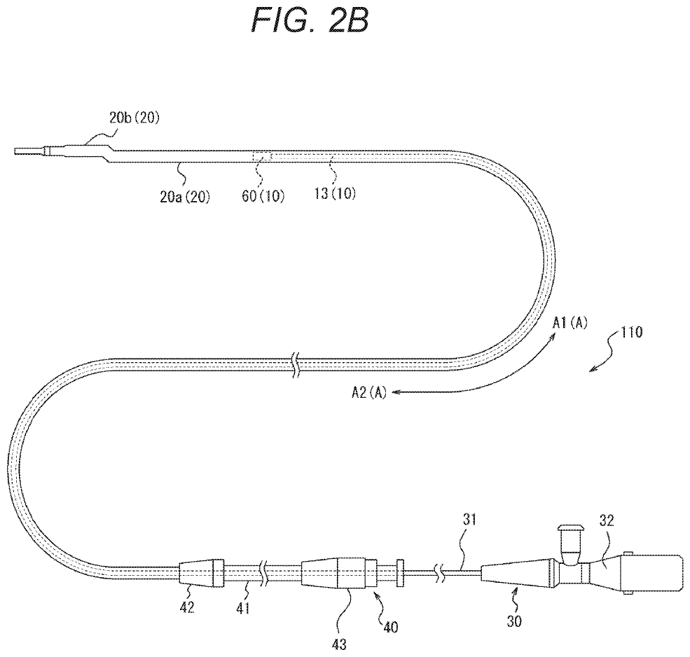

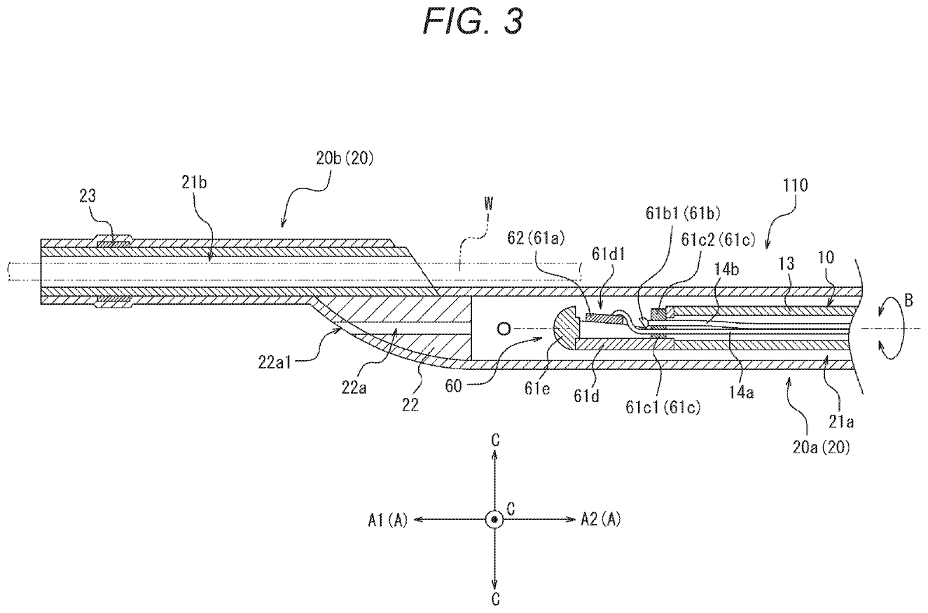

FIGS. 2A and 2B are views illustrating the diagnostic imaging catheter 110 illustrated in FIG. 1 as a single unit. Although details will be described later, FIGS. 2A and 2B illustrate a state where the position of a probe 10 in the catheter longitudinal direction A in a sheath 20 is different. FIG. 3 is a view illustrating an end portion on a distal side (hereinafter, referred to as a “distal end portion”) of the diagnostic imaging catheter 110. For example, the diagnostic imaging catheter 110 is configured to acquire a tomographic image of a lumen of a vessel. More specifically, the diagnostic imaging catheter 110 includes an ultrasound transmitter and receiver 61a for IVUS and an optical transmitter and receiver 61b for OCT/OFDI. As illustrated in FIG. 1, the diagnostic imaging catheter 110 is driven by being connected to the external device 120. More specifically, the diagnostic imaging catheter 110 is connected to a drive unit 120a of the external device 120.

### Diagnostic Imaging Catheter 110

As illustrated in FIGS. 1 to 3, the diagnostic imaging catheter 110 includes a probe 10, an elongated sheath 20, an inner tube 30, and an outer tube 40. Hereinafter, each part of the diagnostic imaging catheter 110 will be described in detail.

### [Probe 10]

As illustrated in FIG. 3, the probe 10 includes an imaging core 60, a drive shaft 13, and an electrical signal line 14a and an optical transmission line 14b extending in the drive shaft 13. The imaging core 60 includes an ultrasound transmitter and receiver 61a, an optical transmitter and receiver 61b, a holding member 61c, a housing 61d, and a protective member 61e.

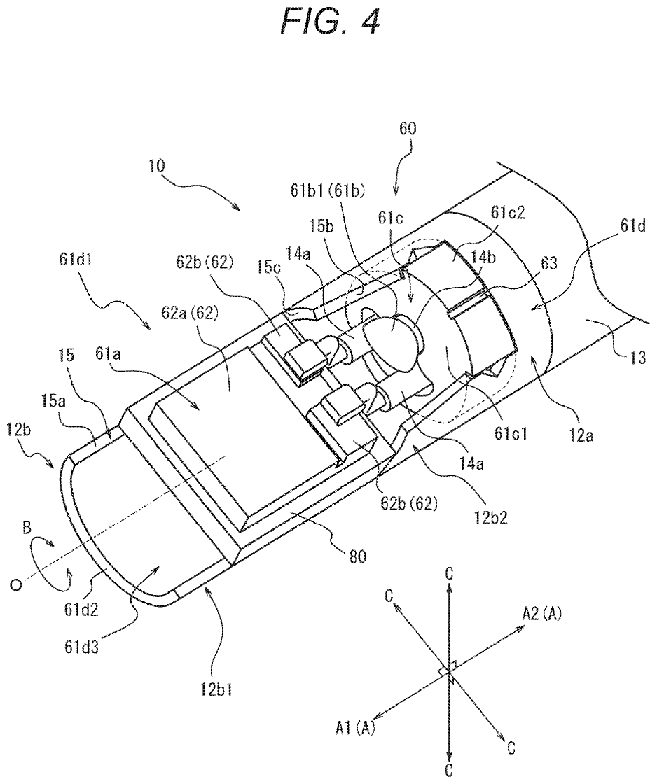

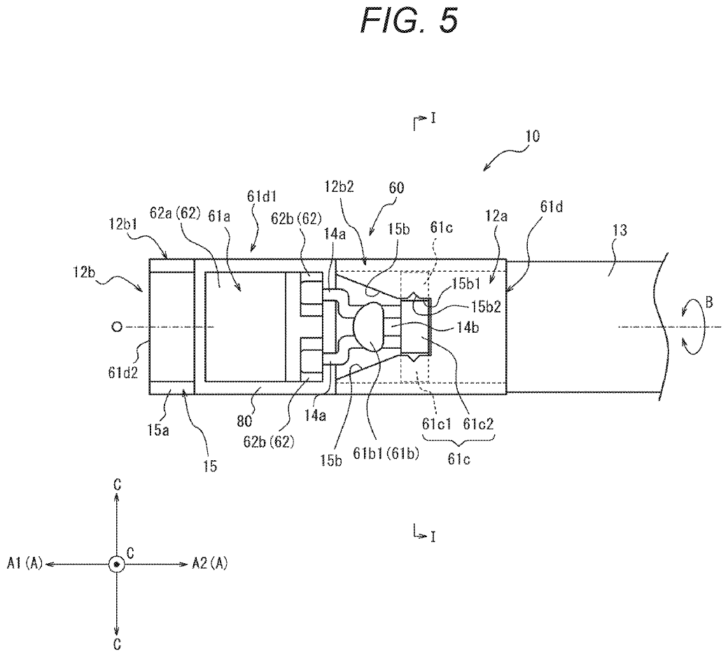

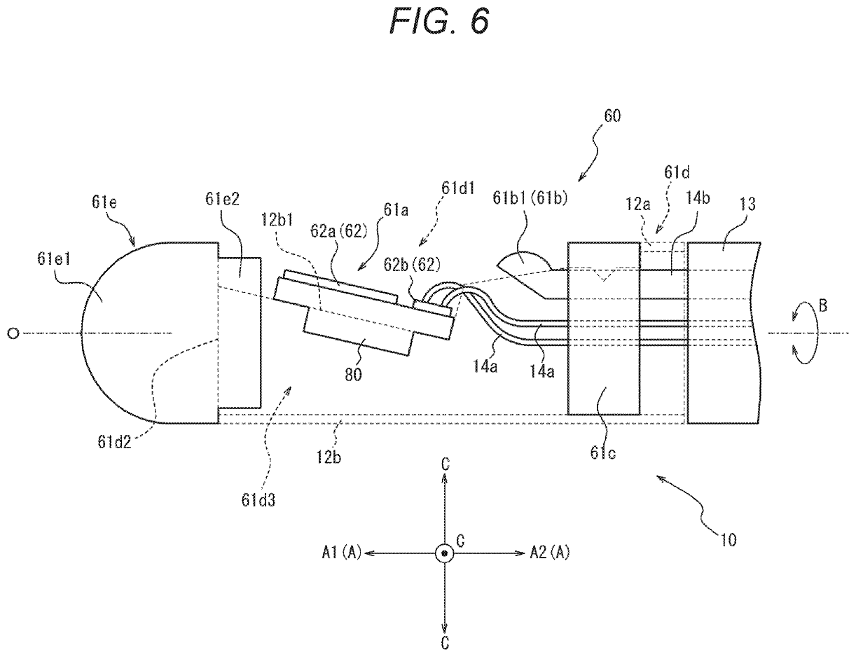

FIG. 4 is a perspective view of the probe 10 in the vicinity of the imaging core 60. FIG. 5 is a top view of the probe 10 in the vicinity of the imaging core 60. In FIGS. 4 and 5, the protective member 61e is not illustrated for convenience of description. FIG. 6 is a view illustrating a positional relationship among the imaging core 60, the drive shaft 13, the electrical signal line 14a, and the optical transmission line 14b in a case where the probe 10 in the vicinity of the imaging core 60 is viewed from a side surface side of the imaging core 60. In FIG. 6, the housing 61d of the imaging core 60 is indicated by a broken line for convenience of description.

As illustrated in FIGS. 4 to 6, the imaging core 60 is connected to the drive shaft 13. More specifically, the imaging core 60 is fixed to the distal end portion of the drive shaft 13. The ultrasound transmitter and receiver 61a of the imaging core 60 includes an ultrasound transducer 62. The ultrasound transducer 62 can transmit an ultrasound wave based on a pulse signal in the lumen in the living body and receive the ultrasound wave reflected from a biological tissue around the lumen. The ultrasound transducer 62 includes a main body portion 62a and an electrode 62b. The main body portion 62a includes a piezoelectric element. The piezoelectric element includes, for example, a piezoelectric body made of ceramics, quartz, or the like. The ultrasound transmitter and receiver 61a can transmit and receive ultrasound waves by means of the ultrasound transducer 62. The ultrasound transmitter and receiver 61a is located on the distal side relative to an optical element 61b1 (to be described later) of the optical transmitter and receiver 61b.

The optical transmitter and receiver 61b can continuously transmit the transmitted light to the lumen and continuously receive the light reflected from the biological tissue around the lumen. The optical transmitter and receiver 61b includes the optical element 61b1. The optical element 61b1 is connected to the distal end portion of the optical transmission line 14b and has a lens function of condensing light and a reflection function of reflecting light.

The optical element 61b1 is a ball lens including a planar portion inclined with respect to the catheter longitudinal direction A and a spherical portion. The planar portion is provided with a reflective coat that reflects light propagating from the optical transmission line 14b. The constituent material of the reflective coat is not particularly limited as long as it can reflect light, and examples thereof include aluminum. The light propagating from the optical transmission line 14b is reflected by the planar portion, condensed by the spherical portion, and transmitted to the lumen. The light reflected by the biological tissue around the lumen is condensed by the spherical portion, reflected by the planar portion, and propagated to the optical transmission line 14b. In this manner, the optical transmitter and receiver 61b can transmit and receive the light through the optical element 61b1.

The holding member 61c holds the optical transmission line 14b connected to the optical transmitter and receiver 61b. More specifically, the holding member 61c holds the optical transmission line 14b connected to the proximal side of the optical transmitter and receiver 61b on the proximal side in the catheter longitudinal direction A relative to the optical transmitter and receiver 61b. The holding member 61c may include, for example, a radiopaque marker portion having X-ray opacity. Furthermore, the holding member 61c may be, for example, a radiopaque marker member that entirely has X-ray opacity. The X-ray opacity of the holding member 61c may be achieved by using a material having high X-ray opacity. Specifically, the holding member 61c may contain a material having high X-ray opacity such as platinum, gold, iridium, or tungsten.

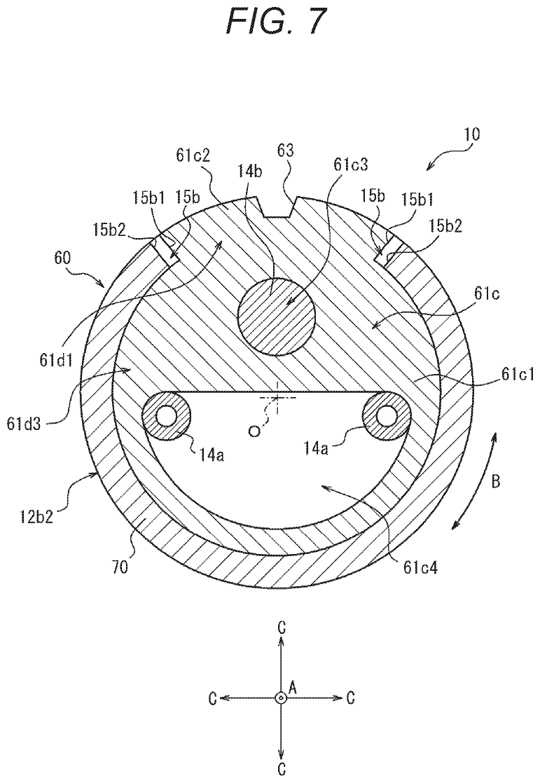

FIG. 7 is a cross-sectional view of the probe 10 in a plane orthogonal to the catheter longitudinal direction A at a position of the holding member 61c. As illustrated in FIG. 7, the holding member 61c is formed with a holding hole 61c3 through which the optical transmission line 14b is inserted in the catheter longitudinal direction A and which sandwiches the optical transmission line 14b with its inner surface, and an insertion hole 61c4 through which the electrical signal line 14a is inserted in the catheter longitudinal direction A. Each of the holding hole 61c3 and the insertion hole 61c4 is a through-hole penetrating the holding member 61c in the catheter longitudinal direction A, but may be, for example, a cutout opening to the outer surface of the holding member 61c in the catheter radial direction C.

The holding member 61c is disposed on the proximal side relative to the optical element 61b1 of the optical transmitter and receiver 61b. The holding member 61c is fixed to the housing 61d. More specifically, the holding member 61c is fixed to the housing 61d in a curved groove-shaped plate portion 12b (to be described later) of the housing 61d. The optical transmission line 14b is held by the inner surface defining the holding hole 61c3 in a state of being inserted into the holding hole 61c3 of the holding member 61c fixed to the housing 61d. Thus, the optical transmitter and receiver 61b connected to the optical transmission line 14b is positioned in the housing 61d.

Furthermore, as illustrated in FIG. 7, the holding member 61c includes a main body 61c1 accommodated in an accommodation space 61d3 (to be described later) of the housing 61d, and a protruding portion 61c2 protruding outward in the catheter radial direction C from the main body 61c1.

The main body 61c1 has a substantially cylindrical outer shape. The holding hole 61c3 and the insertion hole 61c4 described above are formed in the main body 61c1.

The protruding portion 61c2 is positioned relative to the housing 61d so as to be held by edge portions on both sides in the catheter circumferential direction B of an opening portion 61d1 (to be described later) of the housing 61d in the catheter circumferential direction B. This will be described later in detail. Furthermore, the protruding portion 61c2 includes a groove 63 formed on the outer surface in the catheter radial direction C. The groove 63 is formed at a position corresponding to the position, in the catheter circumferential direction B, of the light transmitted from the optical transmitter and receiver 61b toward the outside in the catheter radial direction C. By providing such a groove 63, the alignment of the transmission direction of the light from the optical transmitter and receiver 61b and the transmission direction of the ultrasound wave from the ultrasound transmitter and receiver 61a in the catheter circumferential direction B can be easily performed from the outside by aligning the ultrasound wave transmitted from the ultrasound transmitter and receiver 61a and the groove 63 without actually transmitting the light from the optical transmitter and receiver 61b.

The housing 61d directly or indirectly supports the ultrasound transmitter and receiver 61a, the optical transmitter and receiver 61b, and the holding member 61c. The distal end portion of the drive shaft 13 is connected to the housing 61d. That is, the drive shaft 13 is continuous with the proximal side in the catheter longitudinal direction A relative to the housing 61d. The housing 61d is only required to be integrated with the drive shaft 13. Therefore, the housing 61d may be directly connected to the drive shaft 13 by means of adhesion or the like, or may be indirectly connected to the drive shaft 13 via a connector or the like.

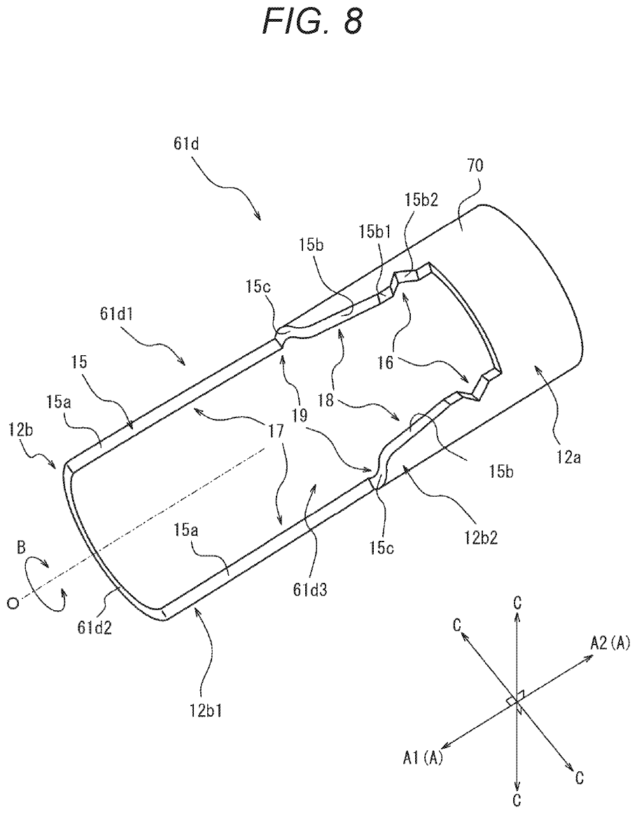

FIG. 8 is a perspective view illustrating the housing 61d. As illustrated in FIGS. 4 to 8, an opening portion 61d1 is formed in the housing 61d. The opening portion 61d1 allows an ultrasound wave transmitted and received by the ultrasound transmitter and receiver 61a and light transmitted and received by the optical transmitter and receiver 61b to pass therethrough. That is, the ultrasound transmitter and receiver 61a can transmit an ultrasound wave based on the pulse signal to the lumen through the opening portion 61d1. Furthermore, the ultrasound transmitter and receiver 61a can receive the ultrasound wave reflected from the biological tissue around the lumen through the opening portion 61d1. Moreover, the optical transmitter and receiver 61b can transmit the light to the lumen through the opening portion 61d1. Furthermore, the optical transmitter and receiver 61b can receive the light reflected from the biological tissue around the lumen through the opening portion 61d1. As illustrated in FIG. 8, the opening portion 61d1 of the housing 61d is formed in a peripheral wall 70 that internally defines the accommodation space 61d3 capable of accommodating the ultrasound transmitter and receiver 61a, the optical transmitter and receiver 61b, and the holding member 61c.

More specifically, as illustrated in FIG. 8, the housing 61d includes a tubular portion 12a and the curved groove-shaped plate portion 12b continuous with the tubular portion 12a on the distal side in the catheter longitudinal direction A. The curved groove-shaped plate portion 12b is formed by extending only a part of the peripheral wall of the tubular portion 12a in the catheter circumferential direction B toward the distal side in the catheter longitudinal direction A. In other words, the tubular portion 12a extends endlessly in the catheter circumferential direction B, whereas the curved groove-shaped plate portion 12b does not extend endlessly in the catheter circumferential direction B. The opening portion 61d1 is formed between both end surfaces 15 of the curved groove-shaped plate portion 12b in the catheter circumferential direction B. That is, the edge portions on both sides of the opening portion 61d1 in the catheter circumferential direction B are constituted by both end surfaces 15 of the curved groove-shaped plate portion 12b in the catheter circumferential direction B.

The tubular portion 12a constitutes a proximal end portion of the housing 61d. The tubular portion 12a is located on the proximal side relative to the ultrasound transmitter and receiver 61a, the optical transmitter and receiver 61b, and the holding member 61c. The drive shaft 13 is connected to the tubular portion 12a of the housing 61d. Furthermore, the curved groove-shaped plate portion 12b constitutes the distal end portion of the housing 61d. That is, the opening portion 61d1 of the housing 61d extends to the distal end 61d2 of the housing 61d.

As illustrated in FIGS. 4, 5, and 7, each of the ultrasound transmitter and receiver 61a and the holding member 61c is positioned in the catheter circumferential direction B relative to the housing 61d by the edge portions on both sides of the opening portion 61d1 of the housing 61d in the catheter circumferential direction B. Therefore, it is possible to easily position the ultrasound transmitter and receiver 61a and the optical transmitter and receiver 61b relative to the housing 61d while maintaining a positional relationship between the transmission direction of the ultrasound wave from the ultrasound transmitter and receiver 61a and the transmission direction of light from the optical transmitter and receiver 61b in a desired state in the catheter circumferential direction B.

More specifically, the curved groove-shaped plate portion 12b includes a distal portion 12b1 having an arc-shaped or substantially U-shaped outer shape in a cross-sectional view orthogonal to the catheter longitudinal direction A, and a proximal portion 12b2 having a substantially C-shaped outer shape in a cross-sectional view orthogonal to the catheter longitudinal direction A. The proximal portion 12b2 is located on the proximal side relative to the distal portion 12b1 in the catheter longitudinal direction A.

The above-described opening portion 61d1 is formed over the entire region where the curved groove-shaped plate portion 12b is located in the catheter longitudinal direction A. However, the width of the opening portion 61d1 in the catheter circumferential direction B varies depending on the position in the catheter longitudinal direction A. Specifically, the width of the opening portion 61d1 in the catheter circumferential direction B at the position of the distal portion 12b1 in the catheter longitudinal direction A is greater than the width of the opening portion 61d1 in the catheter circumferential direction B at the position of the proximal portion 12b2 in the catheter longitudinal direction A. The width of the opening portion 61d1 in the catheter circumferential direction B refers to a central angle of the opening portion 61d1 centered on the central axis O.

The distal portion 12b1 supports the ultrasound transmitter and receiver 61a. Specifically, the ultrasound transmitter and receiver 61a is bridged across the edge portions on both sides of the opening portion 61d1 in the catheter circumferential direction B, and is supported by the edge portions on both sides. As described above, the edge portions on both sides of the opening portion 61d1 in the catheter circumferential direction B are constituted by both end surfaces 15 of the curved groove-shaped plate portion 12b in the catheter circumferential direction B. That is, the ultrasound transmitter and receiver 61a is supported by both end surfaces 15a of the distal portion 12b1, which are part of both end surfaces 15 of the curved groove-shaped plate portion 12b.

More specifically, the distal portion 12b1 supports the ultrasound transmitter and receiver 61a via a backing member 80. The backing member 80 scatters and attenuates the ultrasound wave traveling from the ultrasound transmitter and receiver 61a in a direction opposite to the opening portion 61d1 of the housing 61d. A method for fixing the backing member 80 to the distal portion 12b1 is not particularly limited. The backing member 80 may be fixed to the distal portion 12b1, for example, by means of adhesion using an adhesive. The backing member 80 supports the ultrasound transmitter and receiver 61a such that the ultrasound transmitter and receiver 61a can transmit an ultrasound wave in a direction inclined with respect to the catheter longitudinal direction A. More specifically, the backing member 80 is supported by the end surfaces 15a of the distal portion 12b1 extending obliquely with respect to the catheter longitudinal direction A. Thus, the ultrasound transmitter and receiver 61a supported by the backing member 80 is inclined with respect to the catheter longitudinal direction A. Therefore, the ultrasound transmitter and receiver 61a can transmit the ultrasound wave in a direction inclined with respect to the catheter longitudinal direction A.

Furthermore, the proximal portion 12b2 supports the holding member 61c. More specifically, the holding member 61c includes the main body 61c1 having a substantially cylindrical outer shape, and the protruding portion 61c2 protruding outward in the catheter radial direction C relative to the main body 61c1. The proximal portion 12b2 accommodates the main body 61c1 in the accommodation space 61d3 inside the proximal portion 12b2. The protruding portion 61c2 of the holding member 61c protrudes so as to be fitted into the opening portion 61d1 in a state where the main body 61c1 of the holding member 61c is accommodated in the accommodation space 61d3 inside the proximal portion 12b2. Therefore, the protruding portion 61c2 is restricted from rotating in the catheter circumferential direction B with respect to the housing 61d by the edge portions on both sides in the catheter circumferential direction B of the opening portion 61d1. As described above, the edge portions on both sides of the opening portion 61d1 in the catheter circumferential direction B are constituted by both end surfaces 15 of the curved groove-shaped plate portion 12b in the catheter circumferential direction B. That is, the protruding portion 61c2 of the holding member 61c is restricted from rotating by both end surfaces 15b of the proximal portion 12b2, which are part of both end surfaces 15 of the curved groove-shaped plate portion 12b. As described above, the proximal portion 12b2 supports the holding member 61c.

As described above, the position of the ultrasound transmitter and receiver 61a relative to the housing 61d in the catheter circumferential direction B is determined by both end surfaces 15a of the distal portion 12b1 as the edge portions on both sides of the opening portion 61d1 of the housing 61d in the catheter circumferential direction B. Furthermore, the position of the holding member 61c relative to the housing 61d in the catheter circumferential direction B is determined by both end surfaces 15b of the proximal portion 12b2 as the edge portions on both sides of the opening portion 61d1 of the housing 61d in the catheter circumferential direction B. Therefore, it is possible to easily position the ultrasound transmitter and receiver 61a and the optical transmitter and receiver 61b relative to the housing 61d while maintaining a positional relationship between the transmission direction of the ultrasound wave from the ultrasound transmitter and receiver 61a and the transmission direction of light from the optical transmitter and receiver 61b in a desired state in the catheter circumferential direction B.

More specifically, as illustrated in FIG. 8, both end surfaces 15 of the curved groove-shaped plate portion 12b as the edge portions on both sides of the opening portion 61d1 of the housing 61d in the catheter circumferential direction B include a rotation restricting portion 16, a support portion 17, and a guide portion 18.

The rotation restricting portion 16 is configured to restrict the rotation of the protruding portion 61c2 relative to the housing 61d in the catheter circumferential direction B by holding the protruding portion 61c2 of the holding member 61c from both sides in the catheter circumferential direction B and abutting the protruding portion 61c2. As described above, the rotation restricting portion 16 is constituted by both end surfaces 15b of the proximal portion 12b2 of the curved groove-shaped plate portion 12b.

The support portion 17 is provided such that the ultrasound transmitter and receiver 61a is bridged across the support portion 17, and is configured to support the ultrasound transmitter and receiver 61a. The support portion 17 is located on the distal side in the catheter longitudinal direction A relative to the rotation restricting portion 16. As described above, the support portion 17 is constituted by both end surfaces 15a of the distal portion 12b1 of the curved groove-shaped plate portion 12b. Both end surfaces 15a of the distal portion 12b1 are surfaces extending substantially parallel and included in the same plane. The ultrasound transmitter and receiver 61a is supported in a state of being bridged between both end surfaces 15a of the distal portion 12b1, thereby being positioned so as not to rotate with respect to the housing 61d in the catheter circumferential direction B.

The guide portion 18 is configured such that a distance therebetween in the catheter circumferential direction B gradually decreases from a distal side toward a proximal side in the catheter longitudinal direction A toward the rotation restricting portion 16. The guide portion 18 is located between the rotation restricting portion 16 and the support portion 17 in the catheter longitudinal direction A. The proximal end of the guide portion 18 is continuous with the distal end of the rotation restricting portion 16. Similarly to the rotation restricting portion 16, the guide portion 18 is constituted by both end surfaces 15b of the proximal portion 12b2 of the curved groove-shaped plate portion 12b. When the holding member 61c is fixed to the housing 61d, the protruding portion 61c2 passes through the guide portion 18 and moves to a position of the rotation restricting portion 16 from the distal side toward the proximal side in the catheter longitudinal direction A. At this time, the moving direction of the protruding portion 61c2 is guided by coming into contact with the guide portion 18. Therefore, by providing the guide portion 18, the protruding portion 61c2 can be easily moved to a position of the rotation restricting portion 16.

As described above, each of the rotation restricting portion 16 and the guide portion 18 is constituted by both end surfaces 15b of the proximal portion 12b2 of the curved groove-shaped plate portion 12b in the catheter circumferential direction B. More specifically, a portion of the end surfaces 15b of the proximal portion 12b2 in the catheter circumferential direction B, which constitutes the rotation restricting portion 16, includes: a restricting portion 15b1 that extends substantially parallel to the catheter longitudinal direction A and is capable of restricting the rotation of the protruding portion 61c2 relative to the housing 61d in the catheter circumferential direction B by coming into contact with the protruding portion 61c2 of the holding member 61c; and a recessed portion 15b2 recessed from the restricting portion 15b1 in the catheter circumferential direction B. By providing such a recessed portion 15b2, it is easy to fill the accommodation space 61d3 inside the proximal portion 12b2 with an adhesive or the like through the recessed portion 15b2. That is, with the end surfaces 15b of the proximal portion 12b2, while the rotation of the protruding portion 61c2 of the holding member 61c in the catheter circumferential direction B is restricted by the restricting portion 15b1, the accommodation space 61d3 inside the proximal portion 12b2 is easily filled with the adhesive for fixing the main body 61c1 to the proximal portion 12b2 through the recessed portion 15b2. The end surfaces 15b preferably include both the restricting portion 15b1 located on the distal side of the recessed portion 15b2 and the restricting portion 15b1 located on the proximal side of the recessed portion 15b2. As described above, a space as a filling port of the adhesive in the recessed portion 15b2 can be secured more reliably.

Furthermore, as described above, the support portion 17 is constituted by both end surfaces 15a of the distal portion 12b1 in the catheter circumferential direction B. More specifically, both end surfaces 15a of the distal portion 12b1 extend obliquely with respect to the catheter longitudinal direction A such that the width of the opening portion 61d1 in the catheter circumferential direction B gradually increases from the distal side toward the proximal side in the catheter longitudinal direction A. However, for example, both end surfaces 15a of the distal portion 12b1 may extend substantially parallel to the catheter longitudinal direction A.

The edge portions on both sides of the opening portion 61d1 of the housing 61d in the catheter circumferential direction B include an abutment portion 19 in addition to the above-described rotation restricting portion 16, support portion 17, and guide portion 18. The abutment portion 19 faces the distal side in the catheter longitudinal direction A, and is configured to abut the ultrasound transmitter and receiver 61a supported by the support portion 17. The ultrasound transmitter and receiver 61a is positioned in the catheter longitudinal direction A by being abutted against the abutment portion 19. The abutment portion 19 is constituted by step surfaces 15c that are continuous with the end surfaces 15a of the distal portion 12b1 in the catheter circumferential direction B and the end surfaces 15b of the proximal portion 12b2 in the catheter circumferential direction B, among the end surfaces 15 of the curved groove-shaped plate portion 12b.

The housing 61d may be formed by, for example, machining a metal mass or by metallic powder injection molding (MIM).

The protective member 61e is attached to the housing 61d in a state of covering the distal end 61d2 of the housing 61d. The protective member 61e includes a main body portion 61e1 having a substantially hemispherical outer shape, and a fixing portion 61e2 that is continuous with the proximal side of the main body portion 61e1 and fixed to the distal portion 12b1 of the curved groove-shaped plate portion 12b. The fixing portion 61e2 may be, for example, a cylindrical portion fixed to a recessed inner surface of the distal portion 12b1 by means of adhesion or the like. By providing the protective member 61e, friction and catching with the inner surface of the sheath 20 can be suppressed, and damage to the sheath 20 due to the housing 61d can be suppressed. The protective member 61e may be made of, for example, a resin material. Furthermore, the protective member 61e may include, for example, a coil. Furthermore, the protective member 61e may be, for example, a resin tube member that is externally fitted onto and fixed to the distal portion 12b1 of the curved groove-shaped plate portion 12b of the housing 61d.

As illustrated in FIGS. 2A, 2B, and 3, the drive shaft 13 extends through the inside of the sheath 20, the inner tube 30, and the outer tube 40. As described above, the distal end portion of the drive shaft 13 is connected to the housing 61d of the imaging core 60. The proximal end of the drive shaft 13 is held by a hub 32 (to be described later) that constitutes the proximal end of the inner tube 30.

As illustrated in FIGS. 3 to 6, the electrical signal line 14a is connected to the ultrasound transmitter and receiver 61a. As illustrated in FIGS. 3 and 6, the electrical signal line 14a extends through the drive shaft 13. The electrical signal line 14a electrically connects the ultrasound transmitter and receiver 61a of the imaging core 60 and the external device 120 (see FIG. 1). A plurality of (e.g., two) electrical signal lines 14a are provided, and as illustrated in FIGS. 4 to 6, each electrical signal line 14a is connected to the electrode 62b of the ultrasound transmitter and receiver 61a of the imaging core 60. The connection between each electrical signal line 14a and the electrode 62b may be performed, for example, by soldering. The plurality of electrical signal lines 14a may be configured by, for example, a twisted pair cable in which two electrical signal lines 14a are twisted together. Each electrical signal line 14a can be a flexible fine wire member having an outer diameter of more than 0 mm and 0.1 mm or less and having flexibility. Each electrical signal line 14a can be configured by, for example, a conductive wire and a covering material that is formed of an insulating material and covers the periphery of the conductive wire.

As illustrated in FIGS. 3 and 6, the optical transmission line 14b extends in the drive shaft 13. The optical transmission line 14b optically connects the optical transmitter and receiver 61b of the imaging core 60 and the external device 120 (see FIG. 1). As illustrated in FIGS. 3 to 6, the optical transmission line 14b is connected to the optical element 61b1 of the optical transmitter and receiver 61b of the imaging core 60. The optical transmission line 14b includes, for example, an optical fiber as an optical transmission path.

### [Sheath 20]

The sheath 20 is an elongated member that is inserted into a lumen such as a vessel. As illustrated in FIGS. 2A, 2B, and 3, the sheath 20 includes a main body portion 20a and a guidewire insertion portion 20b. A first hollow portion 21a is defined inside the main body portion 20a. A second hollow portion 21b is defined in the guidewire insertion portion 20b. The probe 10 is accommodated in the first hollow portion 21a of the main body portion 20a. The probe 10 can advance and retract in the catheter longitudinal direction A in the first hollow portion 21a. A guidewire W can be inserted into the second hollow portion 21b of the guidewire insertion portion 20b. As illustrated in FIG. 3, the guidewire insertion portion 20b may be provided with a radiopaque marker portion 23 having X-ray opacity. The radiopaque marker portion 23 can be formed of, for example, a metal pipe or a metal coil made of a metal with high X-ray opacity, such as platinum, gold, iridium, or tungsten. Furthermore, as illustrated in FIG. 3, the tubular guidewire insertion portion 20b is adjacent to the distal end portion of the tubular main body portion 20a so as to be parallel to each other. The main body portion 20a and the guidewire insertion portion 20b may be formed by joining different tube members to each other by thermal fusion or the like.

Furthermore, a communication hole 22a1 for communicating between the inside and the outside of the first hollow portion 21a is formed at the distal end portion of the main body portion 20a. Furthermore, a reinforcing member 22 for firmly joining and supporting the guidewire insertion portion 20b is provided at the distal end portion of the main body portion 20a. The reinforcing member 22 is formed with a communication passage 22a that communicates between the inside of the first hollow portion 21a disposed on the proximal side relative to the reinforcing member 22 and the communication hole 22a1. However, the reinforcing member 22 may not be provided at the distal end portion of the main body portion 20a.

The communication hole 22a1 is a priming liquid discharge hole for discharging a priming liquid. When the diagnostic imaging catheter 110 is used, a priming process of filling the main body portion 20a of the sheath 20 with the priming liquid is performed. At the time of performing the priming process, the priming liquid is released from the communication hole 22a1 to the outside, and gas such as air can be discharged from the inside of the main body portion 20a of the sheath 20 together with the priming liquid.

The sheath 20 and the reinforcing member 22 are preferably formed of a flexible material, but the material is not particularly limited. Examples of the constituent material include various thermoplastic elastomers such as styrene-based, polyolefin-based, polyurethane-based, polyester-based, polyamide-based, polyimide-based, polybutadiene-based, trans-polyisoprene-based, fluororubber-based, and chlorinated polyethylene-based elastomers, and a combination of one or more of these (a polymer alloy, a polymer blend, a laminate, or the like) can also be used. Furthermore, a hydrophilic lubricating coating layer exhibiting lubricity when wetted may be disposed on the outer surface of the sheath 20.

### [Inner Tube 30 and Outer Tube 40]

The inner tube 30 accommodates the proximal end portion of the drive shaft 13 and is movable together with the drive shaft 13 within the outer tube 40. As illustrated in FIGS. 1, 2A, and 2B, the inner tube 30 includes an inner tube main body 31 and a hub 32. The inner tube main body 31 is inserted into the outer tube 40 so as to advance and retract. The hub 32 is connected to the proximal side of the inner tube main body 31.

As illustrated in FIGS. 1, 2A, and 2B, the outer tube 40 is fixed to the proximal end portion of the sheath 20. The outer tube 40 includes an outer tube main body 41, a distal-side connector 42, and a proximal-side connector 43. The outer tube main body 41 is located radially outside the inner tube main body 31, and the inner tube main body 31 advances and retracts in the outer tube main body 41. The distal-side connector 42 connects the proximal end portion of the main body portion 20a of the sheath 20 and the distal end portion of the outer tube main body 41. The proximal-side connector 43 is fixed to the proximal end portion of the outer tube main body 41.

The drive shaft 13, the electrical signal line 14a, and the optical transmission line 14b of the probe 10 described above extend from the main body portion 20a of the sheath 20 to the hub 32 constituting the proximal end of the inner tube 30 through the outer tube 40 connected to the proximal side of the main body portion 20a.

The probe 10 and the inner tube 30 described above are connected to each other so as to integrally advance and retract in the catheter longitudinal direction A. Therefore, for example, when the inner tube 30 is pushed in the insertion direction A1, the inner tube 30 is pushed into the outer tube 40 in the insertion direction A1. When the inner tube 30 is pushed into the outer tube 40 in the insertion direction A1, the probe 10 connected to the inner tube 30 moves in the insertion direction A1 within the main body portion 20a of the sheath 20. Thus, a pushed-in state illustrated in FIG. 2A is obtained. When the inner tube 30 is pulled in the withdrawal direction A2 from the pushed-in state illustrated in FIG. 2A, the inner tube 30 is pulled out in the withdrawal direction A2 from the inside of the outer tube 40. When the inner tube 30 is pulled out from the inside of the outer tube 40 in the withdrawal direction A2, the probe 10 connected to the inner tube 30 moves in the withdrawal direction A2 within the main body portion 20a of the sheath 20. Then, a retracted state illustrated in FIG. 2B is obtained.

As illustrated in FIG. 2A, when the inner tube 30 is pushed most in the insertion direction A1, the distal end portion of the inner tube 30 reaches the vicinity of the distal-side connector 42 of the outer tube 40. At this time, the imaging core 60 of the probe 10 is located in the vicinity of the distal end of the main body portion 20a of the sheath 20.

### External Device 120

As illustrated in FIG. 1, the external device 120 includes a motor 121 that is a power source for rotating the drive shaft 13 (see FIG. 2A and the like), and a motor 122 that is a power source for moving the drive shaft 13 in the catheter longitudinal direction A. The rotational movement of the motor 122 is converted into axial movement by means of a ball screw 123 connected to the motor 122.

More specifically, the external device 120 includes a drive unit 120a, a control device 120b electrically connected to the drive unit 120a in a wired or wireless manner, and a monitor 120c capable of displaying an image generated by the control device 120b based on an ultrasound wave reception signal and a light reception signal received from the diagnostic imaging catheter 110. The motor 121, the motor 122, and the ball screw 123 are provided in the drive unit 120a. The operation of the drive unit 120a is controlled by the control device 120b. The control device 120b includes, for example, a processor such as a CPU.

The external device 120 is not limited to the configuration illustrated, and may be configured to further include, for example, an external input unit such as a keyboard.

### <<Opening Portion 61d1 of Housing 61d of Imaging Core 60 of Diagnostic Imaging Catheter 110>>

Hereinafter, with reference to FIGS. 3 to 8, further details of the opening portion 61d1 of the housing 61d of the imaging core 60 of the diagnostic imaging catheter 110 will be described.

As described above, the housing 61d supports the ultrasound transmitter and receiver 61a, the optical transmitter and receiver 61b, and the holding member 61c. Furthermore, in the housing 61d, the opening portion 61d1 through which an ultrasound wave transmitted and received by the ultrasound transmitter and receiver 61a and light transmitted and received by the optical transmitter and receiver 61b can pass is formed. As illustrated in FIGS. 3 to 6, the opening portion 61d1 is formed at a position adjacent to the ultrasound transmitter and receiver 61a and the optical transmitter and receiver 61b in the catheter radial direction C.

Here, the opening portion 61d1 extends to the distal end 61d2 of the housing 61d, and the ultrasound transmitter and receiver 61a is configured to pass from the distal end 61d2 of the housing 61d toward the proximal side. That is, a part or all of the ultrasound transmitter and receiver 61a is movable to the support position of the distal portion 12b1 of the curved groove-shaped plate portion 12b of the housing 61d by passing through the opening portion 61d1 from the opening end on the distal side of the opening portion 61d1 toward the proximal side. More specifically, the ultrasound transmitter and receiver 61a is movable from the distal end 61d2 of the housing 61d toward the proximal side through the groove inside the substantially semi-cylindrical distal portion 12b1 and the opening portion 61d1 formed at a position facing the distal portion 12b1 in the catheter radial direction C. As described above, with the configuration in which the opening portion 61d1 extends to the distal end 61d2 of the housing 61d, even when the housing 61d is miniaturized, the ultrasound transmitter and receiver 61a can be moved from the distal end 61d2 of the housing 61d to the proximal side, that is, in the withdrawal direction A2 through the opening portion 61d1, and the ultrasound transmitter and receiver 61a can be disposed at a predetermined support position of the housing 61d.

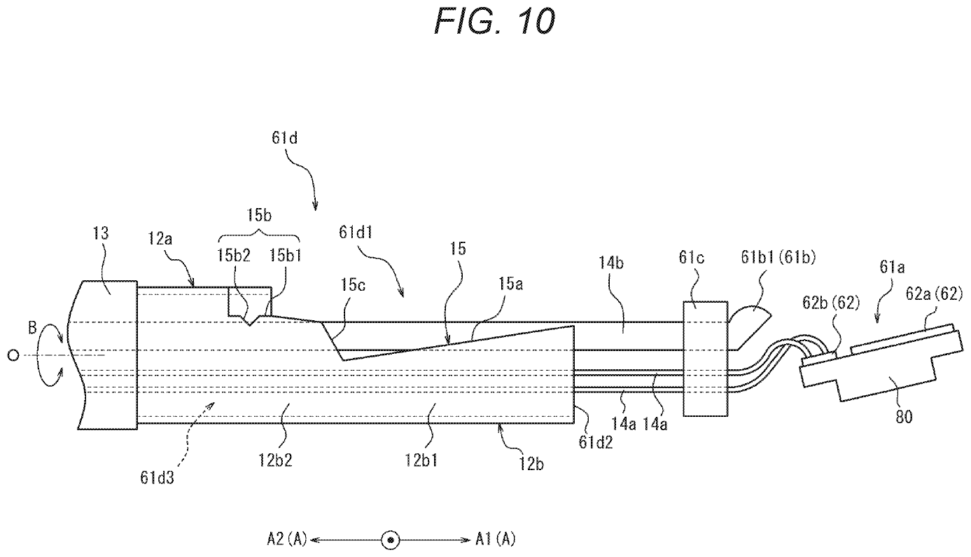

Therefore, even in the diagnostic imaging catheter 110 including the ultrasound transmitter and receiver 61a and the optical transmitter and receiver 61b, the ultrasound transmitter and receiver 61a to which the electrical signal line 14a is connected in advance can be easily attached to the housing 61d while the housing 61d is miniaturized. This will be described later in detail (see FIG. 10).

Furthermore, as illustrated in FIGS. 4 to 7, the imaging core 60 includes the holding member 61c that supports the optical transmission line 14b in the housing 61d. As described above, the holding member 61c is formed with the holding hole 61c3 for inserting and holding the optical transmission line 14b, and the insertion hole 61c4 through which the electrical signal line 14a is inserted. That is, the holding member 61c is fixed to the optical transmission line 14b by inserting the optical transmission line 14b into the holding hole 61c3.

The ultrasound transmitter and receiver 61a is disposed at a position not overlapping with the optical transmitter and receiver 61b in plan view (top view in FIG. 5) in which the housing 61d is viewed from the opening portion 61d1 side, but the present disclosure is not limited to this configuration. The ultrasound transmitter and receiver 61a may be disposed at a position partially overlapping with the optical transmitter and receiver 61b in plan view (top view in FIG. 5) in which the housing 61d is viewed from the opening portion 61d1 side.

Furthermore, as illustrated in FIGS. 4 and 8, the housing 61d includes the curved groove-shaped plate portion 12b at a position where the opening portion 61d1 is formed in the catheter longitudinal direction A. The ultrasound transmitter and receiver 61a is supported by both end surfaces 15a of the distal portion 12b1 of the curved groove-shaped plate portion 12b of the housing 61d in the catheter circumferential direction B. More specifically, in the ultrasound transmitter and receiver 61a, the backing member 80 supporting the ultrasound transmitter and receiver 61a is directly supported by both end surfaces 15a, such that it is indirectly supported by both end surfaces 15a. In this manner, by configuring the ultrasound transmitter and receiver 61a to be supported by both end surfaces 15a of the distal portion 12b1 of the housing 61d, the ultrasound transmitter and receiver 61a is easily positioned in the catheter circumferential direction B relative to the housing 61d. Furthermore, since the backing member 80 is supported by both end surfaces 15a, a support surface that supports the ultrasound transmitter and receiver 61a of the backing member 80 can be widely secured as compared with a configuration in which the backing member 80 is not supported by both end surfaces 15a and is supported only in the groove of the distal portion 12b1. Therefore, a large surface area of the main body portion 62a of the ultrasound transmitter and receiver 61a supported on the support surface of the backing member 80 can be secured.

Furthermore, in a case where the ultrasound transmitter and receiver 61a is configured to be supported by both end surfaces 15a of the distal portion 12b1 of the curved groove-shaped plate portion 12b of the housing 61d, as described above, it is particularly advantageous that the opening portion 61d1 extends to the distal end 61d2 of the housing 61d, and the ultrasound transmitter and receiver 61a is configured to be able to pass from the distal end 61d2 of the housing 61d toward the proximal side. In a case where the distal end portion of the housing 61d is formed in a tubular shape having the same inner diameter and outer diameter as those of the tubular portion 12a, the backing member 80 supported by both end surfaces 15a cannot be retracted into the housing 61d from the tubular distal end portion of the housing 61d to the proximal side. This is because the backing member 80 is caught on the tubular distal end of the housing 61d. Therefore, by providing the opening portion 61d1 having an open distal side described above, the above-described catching of the backing member 80 can be prevented, and the backing member 80 can be retracted into the housing 61d from the distal end 61d2 of the housing 61d to the proximal side.

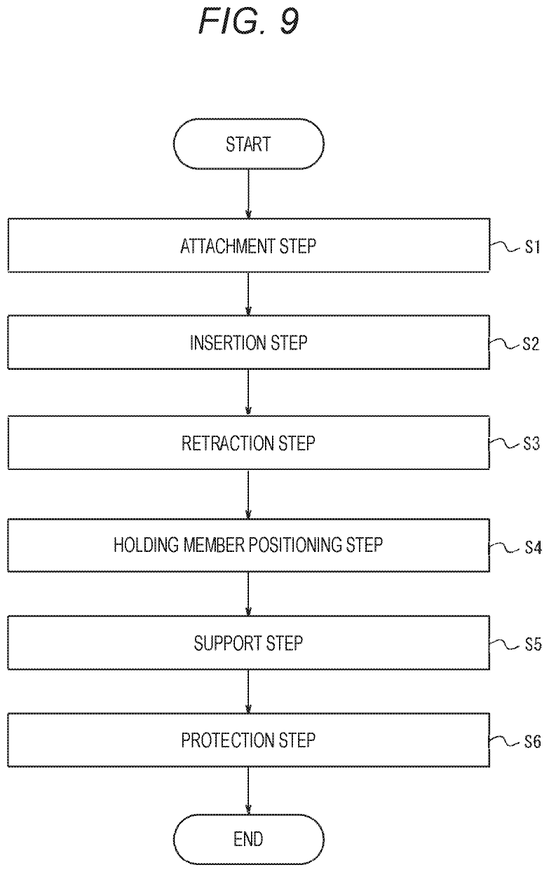

Next, an example of a method of manufacturing the diagnostic imaging catheter 110 will be described with reference to FIGS. 9 and 10. FIG. 9 is a flowchart illustrating an example of the method for manufacturing the diagnostic imaging catheter 110. The manufacturing method illustrated in FIG. 9 includes an attachment step S1, an insertion step S2, a retraction step S3, a holding member positioning step S4, a support step S5, and a protection step S6. FIG. 10 is a view illustrating an outline of the retraction step S3 in which the ultrasound transmitter and receiver 61a and the optical transmitter and receiver 61b are retracted and installed in the housing 61d connected to the distal end portion of the drive shaft 13.

As illustrated in FIG. 10, in the retraction step S3, the ultrasound transmitter and receiver 61a to which the electrical signal line 14a is connected is retracted from the distal end 61d2 of the housing 61d toward the proximal side through the opening portion 61d1 extending to the distal end 61d2 of the housing 61d. In other words, the ultrasound transmitter and receiver 61a is not installed in the housing 61d by moving the ultrasound transmitter and receiver 61a only in the catheter radial direction C, but is moved in the catheter longitudinal direction A through the opening portion 61d1 and installed in the housing 61d. In such a method, since the housing is miniaturized and the optical transmitter and receiver 61b also needs to be installed, even when the housing 61d, in which the installation space of the ultrasound transmitter and receiver 61a is limited, is used, the ultrasound transmitter and receiver 61a can be retracted into the housing 61d in a state where the electrical signal line 14a is connected. Conventionally, in the case of using a housing that is miniaturized and also requires installation of the optical transmitter and receiver 61b, the electrical signal line 14a is disposed in advance in the housing, and then the ultrasound transmitter and receiver 61a is moved in the catheter radial direction C through the opening portion 61d1 and installed in the housing. Since an operation of connecting the ultrasound transmitter and receiver 61a and the electrical signal line 14a is performed in a small housing, an operation of connecting the ultrasound transmitter and receiver 61a and the electrical signal line 14a is difficult. On the other hand, by adopting the above-described retraction step S3, it is possible to improve the ease of attachment of the ultrasound transmitter and receiver 61a and the optical transmitter and receiver 61b to the housing 61d while miniaturizing the housing 61d.

In the retraction step S3, the ultrasound transmitter and receiver 61a to which the electrical signal line 14a is connected may be retracted from the distal end 61d2 of the housing 61d toward the proximal side through the opening portion 61d1 in a state where the optical transmitter and receiver 61b is supported in advance by the housing 61d. However, as illustrated in FIG. 10, the ultrasound transmitter and receiver 61a to which the electrical signal line 14a is connected may be retracted from the distal end 61d2 of the housing 61d toward the proximal side through the opening portion 61d1 together with the optical transmitter and receiver 61b to which the optical transmission line 14b is connected.

Furthermore, as illustrated in FIG. 9, before performing the retraction step S3, the attachment step S1 of attaching the holding member 61c to the optical transmission line 14b and the insertion step S2 of inserting the electrical signal line 14a into the insertion hole 61c4 of the holding member 61c are preferably performed. In the attachment step S1, the optical transmission line 14b is inserted into the holding hole 61c3 of the holding member 61c. Thus, the holding member 61c can be attached to the optical transmission line 14b. Thereafter, in the retraction step S3, it is preferable that the ultrasound transmitter and receiver 61a to which the electrical signal line 14a is connected is retracted from the distal end 61d2 of the housing 61d toward the proximal side through the opening portion 61d1 together with the optical transmitter and receiver 61b to which the optical transmission line 14b is connected and the holding member 61c attached to the optical transmission line 14b. In this manner, in addition to the ultrasound transmitter and receiver 61a to which the electrical signal line 14a is connected and the optical transmitter and receiver 61b to which the optical transmission line 14b is connected, the holding member 61c attached to the optical transmission line 14b and having the electrical signal line 14a inserted through the insertion hole 61c4 can be retracted into the housing 61d together. Therefore, it is possible to further enhance the manufacturing efficiency of the diagnostic imaging catheter 110.

In the holding member positioning step S4, the holding member 61c retracted from the distal end 61d2 of the housing 61d to the proximal side is supported by the proximal portion 12b2 of the curved groove-shaped plate portion 12b of the housing 61d. Specifically, while the main body 61c1 of the holding member 61c is accommodated in the accommodation space 61d3 inside the proximal portion 12b2, the protruding portion 61c2 of the holding member 61c is moved from the guide portion 18 (see FIG. 8) to the rotation restricting portion 16 (see FIG. 8). Thus, while the main body 61c1 of the holding member 61c is accommodated in the accommodation space 61d3, the protruding portion 61c2 of the holding member 61c is restricted from rotating in the catheter circumferential direction B by both end surfaces 15b of the proximal portion 12b2. That is, the holding member 61c can be positioned relative to the housing 61d in a state of not relatively rotating with respect to the housing 61d in the catheter circumferential direction B.

Furthermore, in the support step S5, the ultrasound transmitter and receiver 61a retracted from the distal end 61d2 of the housing 61d to the proximal side is bridged across and supported by both end surfaces 15a of the distal portion 12b1 of the curved groove-shaped plate portion 12b of the housing 61d. As described above, the backing member 80 that supports the ultrasound transmitter and receiver 61a is bridged across and supported by both end surfaces 15a of the distal portion 12b1 of the housing 61d. In this manner, by positioning the ultrasound transmitter and receiver 61a using both end surfaces 15a of the distal portion 12b1 of the curved groove-shaped plate portion 12b of the housing 61d, positioning of the ultrasound transmitter and receiver 61a in the catheter circumferential direction B relative to the housing 61d can be easily performed.

By executing the holding member positioning step S4 and the support step S5 in this manner, relative positioning of the ultrasound transmitter and receiver 61a and the optical transmitter and receiver 61b held by the holding member 61c in the catheter circumferential direction B can be easily performed. Therefore, for example, when the holding member 61c rotates in the catheter circumferential direction B relative to the housing 61d, it is possible to suppress the positional relationship in the catheter circumferential direction B between the transmission direction of the ultrasound wave from the ultrasound transmitter and receiver 61a and the transmission direction of the light from the optical transmitter and receiver 61b from varying from a desired state.

The order of executing the holding member positioning step S4 and the support step S5 described above is not particularly limited. The holding member positioning step S4 and the support step S5 may be executed in this order, or the support step S5 and the holding member positioning step S4 may be executed in this order. Furthermore, the holding member positioning step S4 and the support step S5 may be executed simultaneously.

After completion of the holding member positioning step S4 and the support step S5, the positions of the ultrasound transmitter and receiver 61a and the optical transmitter and receiver 61b relative to the housing 61d are fixed using an adhesive or the like. At this time, filling of an adhesive for fixing the holding member 61c to the housing 61d may be executed through the space in the recessed portion 15b2 (see FIGS. 5, 7, and 8) described above. Since the protruding portion 61c2 is fitted between both end surfaces 15b of the proximal portion 12b2, by providing the recessed portion 15b2 to serve as an adhesive filling port, the adhesive can be more reliably supplied between the main body 61c1 in the proximal portion 12b2 and the inner surface of the proximal portion 12b2.

Thereafter, the protection step S6 of attaching the protective member 61e covering the distal end 61d2 of the housing 61d to the housing 61d is executed.

Here, the retraction step S3 in which the ultrasound transmitter and receiver 61a in a state where the electrical signal line 14a is connected in advance is retracted through the opening portion 61d1 has been described, but the present disclosure is not limited to this manufacturing method. That is, the diagnostic imaging catheter according to the present disclosure may be manufactured by a method for fixing the ultrasound transmitter and receiver 61a to the housing 61d and then connecting the electrical signal line 14a to the ultrasound transmitter and receiver 61a fixed to the housing 61d. Even in such a case, by positioning each of the ultrasound transmitter and receiver 61a and the holding member 61c holding the optical transmitter and receiver 61b via the optical transmission line 14b so as not to rotate in the catheter circumferential direction B relative to the housing 61d, the ultrasound transmitter and receiver 61a and the optical transmitter and receiver 61b can be easily positioned relative to the housing 61d while maintaining the positional relationship in the catheter circumferential direction B between the transmission direction of the ultrasound wave from the ultrasound transmitter and receiver 61a and the transmission direction of the light from the optical transmitter and receiver 61b in a desired state.

The diagnostic imaging catheter according to the present disclosure is not limited to the specific configurations illustrated in the above-described embodiments, and various changes, modifications, and combinations can be made without departing from the scope of the claims.

What is claimed is:

1. A diagnostic imaging catheter comprising:
a drive shaft; and
an imaging core connected to the drive shaft and including:
an ultrasound transmitter and receiver,
an optical transmitter and receiver,
an optical transmission line connected to the optical transmitter and receiver,
a holding member that holds the optical transmission line, and
a housing that supports the ultrasound transmitter and receiver, the optical transmitter and receiver, and the holding member, wherein
the housing has an opening portion through which an ultrasound wave to be transmitted and received by the ultrasound transmitter and receiver and light to be transmitted and received by the optical transmitter and receiver can pass, and
each of the ultrasound transmitter and receiver and the holding member is positioned in a catheter circumferential direction by edge portions on both sides of the opening portion such that the ultrasound transmitter and receiver and the holding member are restrained from rotating relative to the housing in the catheter circumferential direction.

2. The diagnostic imaging catheter according to claim 1, wherein
the opening portion is formed in a peripheral wall that internally defines an accommodation space capable of accommodating the ultrasound transmitter and receiver, the optical transmitter and receiver, and the holding member,
the holding member includes:
a main body accommodated in the accommodation space, and
a protruding portion protruding outward in a catheter radial direction from the main body, and
the edge portions on both sides of the opening portion hold the protruding portion from both sides in the catheter circumferential direction and include a rotation restricting portion that restricts rotation of the protruding portion in the catheter circumferential direction relative to the housing.

3. The diagnostic imaging catheter according to claim 2, wherein
the rotation restricting portion includes:
a restricting portion capable of restricting rotation in the catheter circumferential direction by abutting the protruding portion of the holding member, and
a recessed portion recessed from the restricting portion in the catheter circumferential direction.

4. The diagnostic imaging catheter according to claim 2, wherein
the edge portions on both sides of the opening portion include a support portion across which the ultrasound transmitter and receiver is bridged, the support portion supporting the ultrasound transmitter and receiver.

5. The diagnostic imaging catheter according to claim 4, wherein
the edge portions on both sides of the opening portion include a guide portion in which a distance between the edge portions in the catheter circumferential direction gradually decreases toward the rotation restricting portion from a distal side to a proximal side in a catheter longitudinal direction.

6. The diagnostic imaging catheter according to claim 2, wherein
the protruding portion includes a groove formed on an outer surface in the catheter radial direction.

7. The diagnostic imaging catheter according to claim 1, further comprising:
an electrical signal line connected to the ultrasound transmitter and receiver, wherein the holding member has an insertion hole through which the electrical signal line is inserted.

8. The diagnostic imaging catheter according to claim 7, wherein
the drive shaft is continuous with a proximal side in a catheter longitudinal direction relative to the housing, and
the optical transmission line and the electrical signal line extend within the drive shaft.

9. The diagnostic imaging catheter according to claim 1, wherein
the opening portion extends to a distal end of the housing.

10. The diagnostic imaging catheter according to claim 1, further comprising:
a backing member that supports the ultrasound transmitter and receiver and that is bridged across and supported by the edge portions on both sides of the opening portion, wherein
the backing member is configured to scatter and attenuate an ultrasound wave traveling from the ultrasound transmitter and receiver in a direction opposite to the opening portion.

11. The diagnostic imaging catheter according to claim 10, wherein
the backing member supports the ultrasound transmitter and receiver such that the ultrasound transmitter and receiver is inclined with respect to a catheter longitudinal direction.

12. The diagnostic imaging catheter according to claim 1, wherein
the edge portions on both sides of the opening portion include an abutment portion that faces a distal side in a catheter longitudinal direction and abuts the ultrasound transmitter and receiver, such that the ultrasound transmitter and receiver is positioned in the catheter longitudinal direction.

13. The diagnostic imaging catheter according to claim 1, wherein
the opening portion has a width in the catheter circumferential direction that varies along a catheter longitudinal direction, the width at a position on a distal side in the catheter longitudinal direction being greater than the width at a position on a proximal side in the catheter longitudinal direction.

14. The diagnostic imaging catheter according to claim 1, further comprising:
a protective member attached to the housing so as to cover a distal end of the housing.

15. The diagnostic imaging catheter according to claim 1, wherein
the holding member includes a radiopaque marker portion having X-ray opacity.

16. A method of manufacturing a diagnostic imaging catheter, the method comprising:
retracting an ultrasound transmitter and receiver, to which an electrical signal line is connected, and a holding member that holds an optical transmission line connected to an optical transmitter and receiver, into a housing through an opening portion that extends to a distal end of the housing, from the distal end of the housing toward a proximal side; and
positioning each of the ultrasound transmitter and receiver and the holding member by edge portions on both sides of the opening portion such that the ultrasound transmitter and receiver and the holding member are restrained from rotating relative to the housing in a catheter circumferential direction.

17. The method according to claim 16, further comprising, before the retracting:
attaching the holding member to the optical transmission line by inserting the optical transmission line into a holding hole of the holding member; and
inserting the electrical signal line into an insertion hole of the holding member.

18. The method according to claim 16, wherein
the positioning includes supporting the ultrasound transmitter and receiver by bridging the ultrasound transmitter and receiver across the edge portions on both sides of the opening portion.

19. The method according to claim 16, further comprising:
attaching a protective member to the housing so as to cover the distal end of the housing.

20. A diagnostic imaging apparatus comprising:
a diagnostic imaging catheter; and
an external device to which the diagnostic imaging catheter is connected, wherein
the diagnostic imaging catheter includes:
a drive shaft, and
an imaging core connected to the drive shaft and including:
an ultrasound transmitter and receiver,
an optical transmitter and receiver,
an optical transmission line connected to the optical transmitter and receiver,
a holding member that holds the optical transmission line, and
a housing that supports the ultrasound transmitter and receiver, the optical transmitter and receiver, and the holding member, wherein
the housing has an opening portion through which an ultrasound wave to be transmitted and received by the ultrasound transmitter and receiver and light to be transmitted and received by the optical transmitter and receiver can pass, and
each of the ultrasound transmitter and receiver and the holding member is positioned in a catheter circumferential direction by edge portions on both sides of the opening portion such that the ultrasound transmitter and receiver and the holding member are restrained from rotating relative to the housing in the catheter circumferential direction, and
the external device is configured to:
rotate the drive shaft,
move the drive shaft in a catheter longitudinal direction, and
generate an image based on an ultrasound wave reception signal and a light reception signal received from the diagnostic imaging catheter.
